(19) United States
(12) Patent Application Publication
(10) Pub. No.: US 2024/0269219 A1
(43) Pub. Date: Aug. 15, 2024

(54) PHARMACEUTICAL COMPOSITION AND ITS ANTIVIRAL USE

(71) Applicant: ARONPHARMA SP. Z O.O., Gdansk (PL)

(72) Inventors: Krzysztof LEMKE, Gdynia (PL); Paulina KOWALCZYK, Debica (PL); Barbara KHAIDAKOV, Godkowo (PL); Aleksandra BOJARCZUK, Gdansk (PL); Mariusz BANACH, Sierpc (PL); Wojciech CYPLIK, Chocicza (PL)

(21) Appl. No.: 18/286,161

(22) PCT Filed: Apr. 4, 2022
(86) PCT No.: PCT/PL2022/000021

§ 371 Date: Oct. 9, 2023

(30) Foreign Application Priority Data
Apr. 2, 2021 (PL) P.437487
Sep. 29, 2021 (PL) P.439058

(51) Int. Cl. A61K 36/73; A61K 36/185; A61P 31/14; A61P 31/16; A61P 37/04

(52) U.S. Cl. CPC A61K 36/73; A61K 36/185; A61P 31/14; A61P 31/16; A61P 37/04

(57) ABSTRACT

The subject of the invention is a pharmaceutical composition and preparation, which contains as an active ingredient a mixture of elderberry fruit extract Sambucus nigra and chokeberry fruit extract Aronia melanocarpa in a ratio of 2: 1 to 1:2. The subject of the invention is also the use of this composition/preparation for the manufacture of dietary supplements for use in supportive therapy in the treatment of diseases with a viral basis for internal use (administered orally).

The subject of the invention is a composition and a preparation comprising extracts of chokeberry (Aronia melanocarpa) and elderberry (Sambucus nigra) fruits and its antiviral and immunostimulating properties.

Sambucus nigra is a plant rich in components with high biological activity, mainly containing polyphenols such as flavonols, phenolic acids, proanthocyanidins and anthocyanins, the presence of which is largely responsible for the antiviral activity of berries (Anton et al., 2013; Sekizawa et al., 2013). Preclinical data to date indicate a significant antiviral effect of elderberry extract (Sambucus nigra L.). This effect was demonstrated against different strains of influenza A virus (H3N2, H1N1, H3N2, H1N1, H5N1) and influenza B virus (Krawitz et al., 2011; Roschek et al., 2009; Zakay-Rones et al., 1995). The flavonoids present in S. nigra L. berries bind to H1N1 virions through haemagglutinin and, once bound, block the ability of the virus to infect host cells. Two compounds responsible for this effect have been identified: 5,7,3′,4′-tetra-Omethylquercetin and 5,7-dihydroxy-4-oxo-2-(3,4,5-trihydroxyphenyl)chroman-3-yl-3,4,5-trihydroxycyclohexanecarboxylate (Roschek et al., 2009). Elderberry lectins also bind to sialic acids present on the host cell membrane thus preventing the virus from attaching to the cell (Zakay-Rones et al., 1995). However, the exact mechanism of the antiviral action of elderberry fruit and its extracted active compounds is still under investigation. It has been suggested that this action may also be due to a neutralizing effect towards neuraminidase (Macdonald 2004; Swaminathan et al., 2013), which allows newly multiplied viruses to be released from the cell. Another proposed mechanism is stimulation of the immune system (Barak et al., 2001). It is noteworthy that we can expect different effects depending on the fraction. In mouse studies it was found that the high molecular fraction, containing polysaccharides has the highest antiviral effect. Administration of this fraction to H1N1 virus-infected mice resulted in lower viral load and increased levels of neutralizing antibodies in bronchoalveolar and lung lavage fluid (Kinoshita et al., 2012). The antiviral effects of elderberry are not only limited to viruses that cause upper respiratory tract infection. Elderberry has also been shown to have potent activity against feline immunodeficiency virus (FIV) as it inhibits the formation of syncytia, and the level of inhibition depends on the concentration of the extract (Uncini Manganelli et al., 2005). Elderberry extract has also been shown to completely inhibit replication of four HSV-1 virus strains, including acyclovir-resistant strains. This effect is independent of whether the extract was administered before, during or after virus adsorption to cells (Morag A et al., 1997). Further, an effect against avian coronavirus was also found. It is postulated that the mechanism of this effect is related to the binding of elderberry lectins directly to the virus (Chen et al., 2014).

Clinical studies, also showed a safe and therapeutic effect of elderberry extract in the treatment of influenza A and B, compared to a placebo group (Zakay-Rones et al., 1995, 2004). Independent clinical studies have confirmed the positive effect of elderberry extract on the course of influenza A and B viral infections (Kong, 2009). In another randomized, double-blind clinical trial, elderberry extract significantly reduced cold symptoms, duration and severity in air travelers (Tiralongo et al., 2016). The results of these studies were summarized in a meta-analysis, where it was concluded that standardized elderberry extract was effective in reducing the total duration and severity of upper respiratory tract symptoms compared to the placebo group. The effect of elderberry supplementation is greater for influenza virus infection than the common cold, but supplementation effectively reduces symptoms regardless of the cause (Hawkins, 2019).

The immunomodulatory effect of elderberry is widely discussed in the literature. Studies indicate an immunostimulatory effect by increasing production of cytokines such as IL-1B, TNF-α, IL-6 and IL-8 in human monocytes compared to LPS-stimulated cells (Barak et al., 2001). Increased secretion of TNF-α, IL-6, IL-8 was also observed in A-549 cells (lung alveolar epithelial cells) compared to LPS-stimulated cells. The immunostimulatory effect was unrelated to the presence of cyanidin-3-glucoside, a compound from the anthocyanin group which is considered to be the main active component of elderberry (Torabian et al., 2019). In a subsequent study, elderberry extract was also shown to attenuate the inflammatory response in LPS-activated macrophages (RAW 264.7 line) by reducing the expression of pro-inflammatory genes (TNF-α, IL-6, COX2, iNOS) and inhibiting the increased production of inflammatory mediators (TNF-α, IL-6, PGE2, NO) (Zielinska-Wasielica et al., 2019). Elderberry extract also showed good efficacy in scavenging free radicals and reduced the formation of reactive oxygen species dose-dependently in keratinocyte cell line HaCaT irradiated with UVB. It also significantly reduced the release of inflammatory cytokines by inhibiting mitogen activated protein kinase/activator protein 1 (MAPK/AP-1) and nuclear factor _{K}B (NF-_{K}B) signalling pathways (Lin et al., 2019).

In the case of chokeberry (Aronia melanocarpa) fruit, there are also reports indicating its antiviral activity. So far, in in vitro models, chokeberry juice has been shown to have activity against various influenza virus strains, including oseltamivir-resistant strains. A dose of 0.125 mg/ml inhibited the development of the tested strains by >60%. Administration of chokeberry extract, as well as extracted ellagic acid and myricetin, to mice infected with recombinant influenza virus resulted in a significant decrease in mortality in these animals. The authors of the study suggest that the effect of chokeberry may be partially explained by an inactivating effect against haemagglutinin (Park et al., 2013). The antiviral effect may also be indicated by the fact that the complex product Bioaron C, containing chokeberries, also showed antiviral activity against a broad spectrum of viruses responsible for upper respiratory tract infections (Glatthaar-Saalmüller et al., 2015).

Chokeberry fruit exhibits health-promoting effects due to its high concentration of polyphenols including anthocyanins and caffeic acid derivatives. Administration of chokeberry extract to LPS-induced ocular choroid inflammation in rats resulted in a reduction in the number of inflammatory cells and a decrease in the levels of NO, PGE2 and TNF-α in the ocular aqueous fluid. In this study, chokeberry extract was also shown to inhibit LPS-induced expression of iNOS and COX-2 in RAW 264.7 cells (Ohgami et al., 2005). Further studies indicate that chokeberry concentrate inhibits TNF-α, IL-6 —and IL-8 production— in LPS-stimulated human monocytes. The extract has also been shown to inhibit NF-_{K}B activation in stimulated macrophages of the RAW 264.7 cell line (Apple et al., 2015). Further, the inhibition of the activation of the classical pathway of the complement system, and decrease of nitric oxide production in LPS-induced RAW 264.7 macrophages were found (Ho et al., 2014). Studies of the anti-inflammatory effects of chokeberry using primary mouse splenocyte cells also showed that chokeberry extract inhibits IL-6 secretion from these cells after LPS stimulation, and increases IL-10 secretion in unstimulated cells (Martin et al., 2014).

Preparations containing S. nigra may produce undesirable effects, particularly related to their cyanogenic glycoside content which may have potentially toxic effects. In addition, these preparations may cause adverse gastrointestinal symptoms such as nausea, diarrhea, abdominal pain (Ulbricht et al., 2014). Therefore, it is necessary to standardize elderberry extract and use the lowest possible effective doses. This will help to obtain the best biological activity and minimize side effects.

So far, there is no complex preparation on the market containing standardized elderberry and chokeberry extracts with proven immunomodulatory and antiviral activity. Therefore, it is reasonable to work towards obtaining a combination of chokeberry and elderberry extracts in an appropriate standardization. This will result in consuming a smaller portion and maintaining an optimal concentration of the desired active compounds present in these fruits, what in turn, will translate into a better antiviral effect.

Invention WO2009059218, discloses a method of treating at least one symptom of metabolic syndrome (advantageously diabetes) in an individual and a dedicated composition containing a therapeutically effective amount of a metabolic syndrome modifying agent derived from a berry containing an anthocyanin-rich extract (advantageously elderberry or chokeberry or currant); and a pharmaceutically acceptable carrier.

Another invention WO2005110404 discloses a method of increasing the cytotoxic activity of a chemotherapeutic agent against an abnormal cell proliferation disorder in a patient, comprising administration of an effective amount of the chemotherapeutic agent in combination with an effective cytotoxicity-enhancing amount of an antioxidant-rich berry extract, a method of reducing toxicity of a chemotherapeutic agent in normal cells of a patient undergoing chemotherapy, comprising administration of an antioxidant-rich berry extract before, with or after the chemotherapeutic agent, and a method of increasing the therapeutic index of a chemotherapeutic agent administered to treat abnormally proliferating cells, comprising administration of an antioxidant-rich berry extract before, with or after the chemotherapeutic agent. Advantageously, mentioned abnormal cell proliferation is colon cancer and mentioned chemotherapeutic agent is 5-fluorouracil. Whereas mentioned berry extract is selected from chokeberry extract, raspberry extract, blueberry extract, blackberry extract, cranberry extract, bilberry extract, blackcurrant extract, cherry extract, elderberry extract, grape extract, kiwi extract, strawberry extract or any combination thereof.

The purpose of the invention is to prepare a suitable composition comprising extracts of chokeberry fruit (Aronia melanocarpa) and elderberry fruit (Sambucus nigra) with antiviral and immunostimulatory properties for oral use.

The subject of the invention is a pharmaceutical composition for stimulating immunity and/or enhancing immunity, which comprises:
a mixture of elderberry fruit extract Sambucus nigra and chokeberry fruit extract Aronia melanocarpa,
where a mixture of elderberry fruit extract Sambucus nigra and chokeberry fruit extract Aronia melanocarpa contains anthocyanins 10-30% and polyphenols 15-50%,
the ratio of extracts of elderberry Sambucus nigra and chokeberry Aronia melanocarpa in the composition is 2:1 to 1:2.

The composition comprises a pharmaceutical excipient or diluent or carrier.

The composition is for oral administration, preferably tablets, capsules, solution.

The composition as defined above for use in the manufacture of food supplements for use in supportive therapy for the treatment of viral diseases.

The composition is applicable for use in influenza and/or influenza-like infections, favorably against influenza A/H1N1 virus.

The composition has an immunostimulatory properties.

A preparation comprising a composition as defined above for antiviral use, advantageously against a human betacoronavirus.

The preparation is applied orally.

The preparation is used as a food supplement.

As used above and in the patent description and claims, the term has the following meaning:
Extract— means the same/alternative term as ‘extract’.
Composition— means a term synonymous/alternative with the term “preparation”.

### BRIEF DESCRIPTION OF THE FIGURES

FIG. 1— presents a block diagram of the manufacturing process of the composition according to the invention.

FIG. 2— presents the chromatogram of the chokeberry extract.

FIG. 3— presents the chromatogram of elderberry extract.

FIG. 4— presents the chromatogram of the composition according to the invention.

FIG. 5— presents the effect of chokeberry (A), elderberry (B), and composition according to the invention (C) extracts on HCoV-OC43 virus replication at a dose of 100 TCID50 (median of three independent experiments with three replicates, confidence interval 95%). The results of the microscopic assay overlapped with those of the immunoperoxidase assay. Results were analyzed by Welch's t test using GraphPad Prism 9.1.0.

FIG. 6— presents the effect of the composition according to the invention on the binding of the recombinant hACE2 protein to the SARS-COV2 S virus protein S receptor binding domain (RBD) in a competitive ELISA.

FIG. 7— presents the metabolic activity of PBMC cells cultured in the presence of extracts. The designation “invention”— means the composition according to the present invention.

FIG. 8— presents the concentration of IL-6 in supernatants from PBMC cell cultures in the presence of extracts. The designation “invention”— means the composition according to the present invention.

FIG. 9— presents the concentration of TNF-α in supernatants from PBMC cell cultures in the presence of extracts. The designation “invention”— means the composition according to the present invention.

FIG. 10— presents the antiviral activity of elderberry extract against human influenza A/H1N1 virus. Virus replication inhibitory concentration of 50% (IC₅₀): 122.5 μg/ml.

FIG. 11— presents the antiviral activity of chokeberry extract against human influenza A/H1N1 virus. Virus replication inhibition concentration of 50% (IC₅₀): 102.3 μg/ml.

FIG. 12— presents the antiviral activity of the composition according to the invention 25% [μg/ml] against human influenza A/H1N1 virus. Virus replication inhibition concentration of 50% (IC₅₀): 116 μg/ml.

OSV— Positive control— anti-influenza drug oseltamivir (30 μg/ml).

The invention is illustrated by the following examples of implementation, which do not constitute a limitation thereof.

### EXAMPLE 1

### Process of Producing Elderberry, Chokeberry Fruit Extracts and Composition According to the Invention

The preparation according to the invention is made of elderberry and chokeberry fruits extracts, standardized for anthocyanins and polyphenols content.

The production of standardized extracts is carried out as follows:

The plant material (elderberry or chokeberry fruits, respectively) is placed in extractors and subjected to an extraction process using a selective water-based extraction solvent. The obtained extract is separated from the raw material and filtered.

The filtered extract is subjected to a purification process, directed to a column packed with a selective adsorbent resin. After the adsorption process the column bed is washed with water and then the adsorbed extract is eluted with ethyl alcohol. From the resulting eluate containing concentrated anthocyanins and other polyphenolic compounds, the solvent is removed by evaporation under reduced pressure.

The anthocyanin and polyphenol content of the concentrated eluate is determined and standardized to a specific content using a carrier and then spray-dried.

The preparation according to the invention is obtained by mixing elderberry and chokeberry fruit extracts obtained by the above method in appropriate proportions. In the obtained preparation, the determination of the anthocyanins and polyphenols content is carried out and the compatibility of the anthocyanin profile is confirmed using high performance liquid chromatography (HPLC).

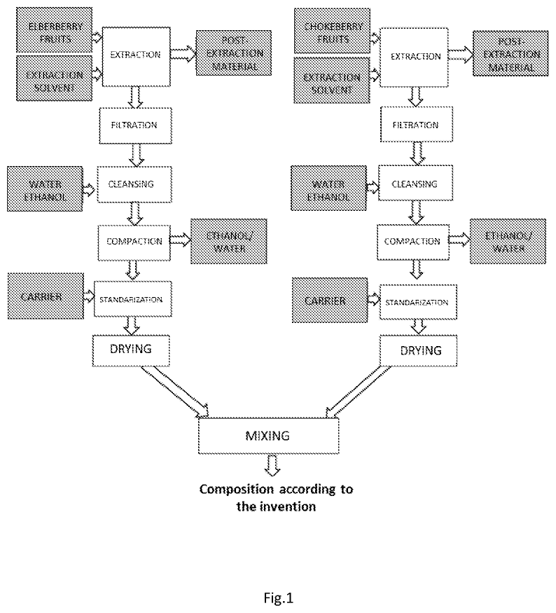

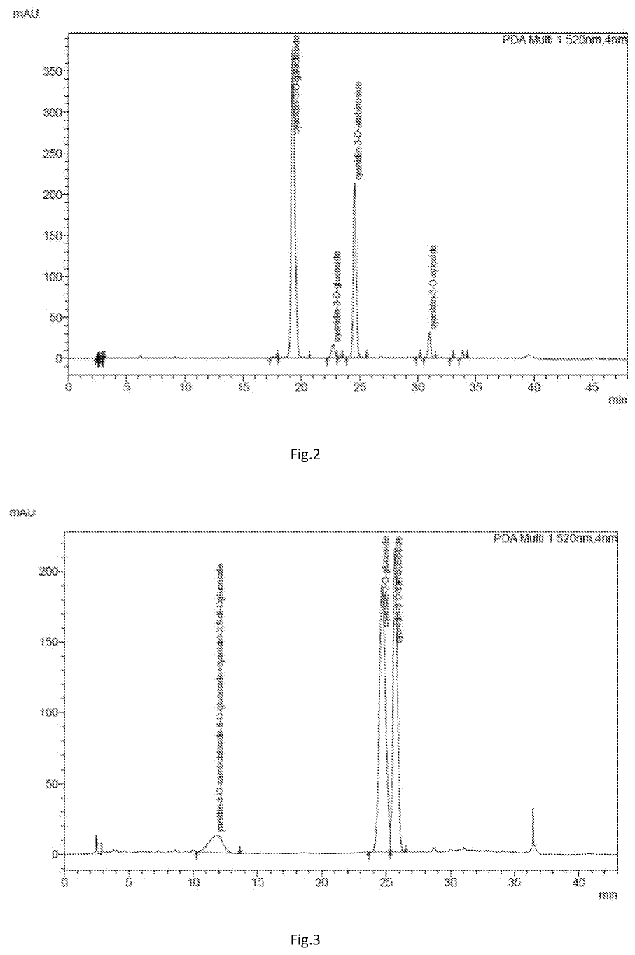

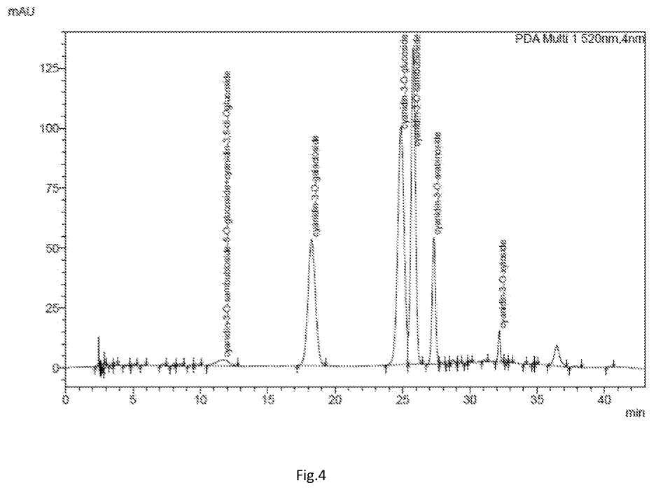

FIG. 1-4.

### EXAMPLE 2

Immunostimulating Effect of Extracts from Elderberry, Chokeberry Fruits and the Composition According to the Invention

### Research Methodology

### Tested Extracts

A standardized extract of elderberry fruits (Sambucus nigra), a standardized extract of chokeberry fruits (Aronia melanocarpa) and a composition according to the invention comprising a standardized extract of chokeberry and a standardized extract of elderberry fruits. Stock solutions of the extracts at a concentration of 100 mg/mL were prepared in 40% DMSO and then brought to a concentration of 250 μg/mL in the culture medium.

### Cells Used for Experiments

Blood mononuclear cells (PBMC; peripheral blood mononuclear cells). PBMC cells were isolated from leukocyte-platelet confluents obtained from healthy blood donors according to a standard procedure (Bulent et al., 2018) using a density gradient with Ficoll (1.077 g/L). Cells were cultured in RPMI-1640 culture medium supplemented with 10% FBS and 100 U/ml penicillin, 100 μg/ml streptomycin.

### Assessment of the Metabolic Activity of Cells— MTT Test

The MTT test is based on the ability of the enzyme mitochondrial dehydrogenase to convert the water-soluble tetrazolium salt to formazan. After dissolving formazan crystals in DMSO, a coloured solution is produced, the intensity of which is measured spectrophotometrically at 570 nm. The amount of coloured, reduced MTT is proportional to the oxidative activity of the cell's mitochondria and, under strict experimental conditions, to the number of living cells in the population. 1×106 PBMC cells were cultured for 24 h in the presence of 250 μg/mL tested extracts. Cells cultured in culture medium were considered as control. After 24 h of incubation, cells were centrifuged at 500×g, 5 min, room temperature. After centrifugation, supernatants (500 μL) were collected for evaluation of cytokine production by ELISA. To the remaining medium with cells, 50 μL of MTT solution (5 mg/mL) was added and incubated for 3 h. After this time, cells were centrifuged at 500×g 5 min, room temperature, supernatant was removed and 750 μL of DMSO was added. Absorbance at 570 nm was read after 10 min.

### Evaluation of the Immunostimulatory Effect

Cytokines were determined in supernatants collected from PBMC cell cultures in the presence of extracts and from control cell cultures without extracts. Commercially available ELISA kits— DuoSet. ELISA Development Systems, R&D—were used to assess cytokine concentrations.

### Results

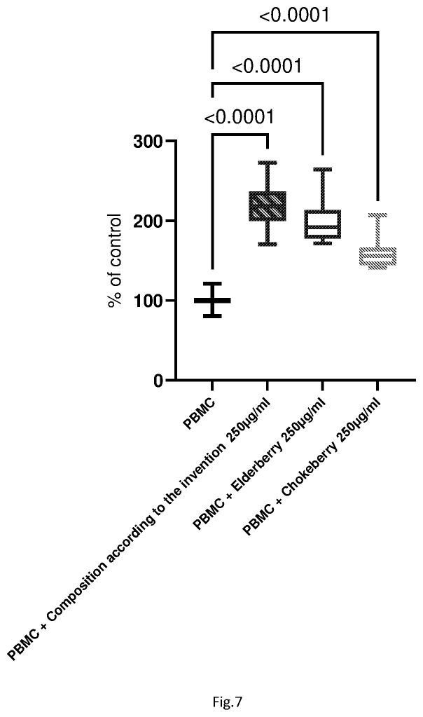

MTT assay showed that the tested extracts were not cytotoxic to PBMC cells at a concentration of 250 μg/mL. An increase in the metabolic activity of PBMC cells incubated with the extracts was evident compared to control cells. The results of MTT analysis as relative values of metabolic activity in cells exposed to the extracts compared to the control group are shown in FIG. 7.

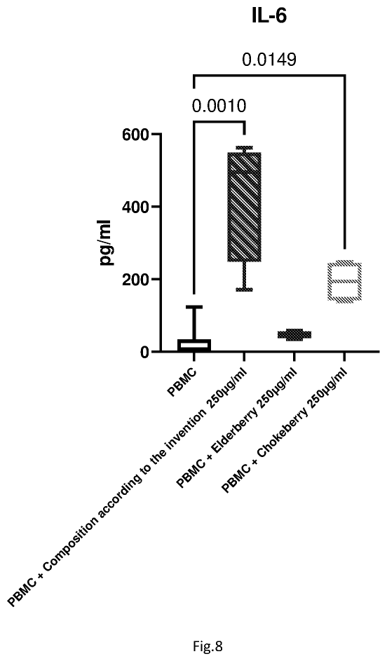

Analysis of the supernatants (FIG. 8-9) showed a significant increase in the production of IL-6 [495.5 μg/mL (171-562.6)] and TNF-α [1337 μg/mL (888-1484)] by PBMC cells after incubation with the composition according to the invention compared to control PBMC cells [IL-6: 4.7 μg/mL (1.17-123); TNF-α: 0.9 μg/mL (0-23.5), respectively]. Standardized chokeberry extract also increased IL-6 (194.1 μg/mL; 138.9-247.9) and TNF-α [1140 μg/mL (1031-1207)] production by PBMC cells. Cells cultured in the presence of standardized elderberry fruit extract showed no significant increase in cytokine production.

### Final Conclusions

1. The composition according to the invention as well as the standardized elderberry and chokeberry extracts do not exhibit cytotoxic activity against PBMC cells at a concentration of 250 μg/mL. At the stated concentration, the tested extracts increase the metabolic activity of PBMC cells.

2. The composition according to the invention has an immunostimulatory effect on PBMC cells resulting in IL-6 and TNF-α production.

3. The immunostimulating effect of the composition according to the invention is stronger than that of elderberry and chokeberry extracts alone.

### EXAMPLE 3

### Antiviral Activity of Elderberry, Chokeberry Extracts and the Composition According to the Invention

### Research Methodology

### Tested Extracts

Standardized extract of elderberry fruits (Sambucus nigra), standardized extract of chokeberry fruits (Aronia melanocarpa) and a composition according to the invention comprising standardized extract of chokeberry and standardized extract of elderberry fruits. A stock solution concentration (100 mg/ml) was prepared in 40% DMSO freshly before each experiment. Subsequently, the extracts were diluted in culture medium to appropriate concentrations. The final DMSO concentration in cells was ≤5% (non-toxic concentration for cells).

### Cell Lines

Canine normal kidney cell line, MDCK (NBL-2)(ATCC CCL-185). Cells were cultured in EMEM medium supplemented with 5% FBS. For influenza virus culture, EMEM medium with 1 mM HEPES, 0.125% BSA fraction V and 1 μg/ml trypsin treated TPCK was used.

### Test Virus

Human influenza A virus, A/H1N1 (Influenzavirus A, strain A/PR/8/34; ATCC-VR-1469TM, Orthomyxoviridae, sheath RNA).

### Cell Viability Test

The CellTiter-Glo. Luminescent Cell Viability Assay was used to assess the cytotoxicity of the extracts against MDCK cells. The CellTiter-Glo. Luminescent Cell Viability Assay is a method for determining the number of viable cells based on the quantification of ATP, an indicator of metabolically active cells. A 24 h culture of MDCK cells with >90% confluence was incubated with tested extracts in the concentration range of 1-5000 μg/ml for 72 h under 37° C. and 5% CO₂.

The control consisted of cells treated with culture medium only. After the set incubation time, 100 μl of CellTiter-Glo. reagent was added to 100 μl of medium including cells, shaken for 2 min on an orbital shaker, incubated for 10 min at room temperature, and then the luminescence was read.

### Antiviral Assay (AVA)

The antiviral activity (AVA) assay, option: incubation of cells with the tested extracts after A/H1N1 virus infection was carried out using selected non-toxic concentrations of the extracts on monolayer 24-hour MDCK cells with a density of 3×105 cells/ml. For elderberry and chokeberry extracts, concentration range of 20-250 μg/ml was used, for the composition according to the invention, concentration range of 100-250 μg/ml was used. The positive control was the anti-influenza drug oseltamivir (30 μg/ml). The selected concentrations of extracts were applied to MDCK cells after virus infection. It was tested whether the extracts added to cell cultures after infection with influenza virus would have an effect on inhibiting its replication in the cells. The CPE of the virus effect was determined under an inverted microscope on a scale of 0-4 (Table 1).

### Determination of Virus Titre by AVA Test

A/H1N1 titres were determined by the cytopathic effects (CPE) assessment method, i.e. changes in cell morphology under the influence of virus replication according to Table 1, where 0— no virus replication (no CPE), 1— CPE in up to 25% of cells, 2— CPE in 25-50% of cells, 3— CPE in 50-75% of cells and 4— up to 100% of cells affected by CPE.

TABLE 1
Cytopathic effects (CPE).

| | Cytopathic effects |
|---|---|
| CPE | in culture [% cells] |
| 0 | 0 |
| 1 | ≤25 |
| 2 | 25-50 |
| 3 | 50-75 |
| 4 | >75 do 100 |

### Results

Determination of Non-Toxic Concentrations of Extracts from Chokeberry, Elderberry and Composition According to the Invention

Elderberry extract showed a complete lack of toxicity or a slight cytotoxic activity in the concentration range 1-1000 μg/ml. Starting from a concentration of 2500 μg/ml, the elderberry extract showed high toxicity to the tested cells. Chokeberry extract showed no or little cytotoxic activity (viability >75%) against MDCK cells in the concentration range 1-500 μg/ml. Starting from an extract concentration of 1000 μg/ml, cell viability decreased to 30%. The composition according to the invention showed a complete lack of toxicity, or little cytotoxicity (viability >75%) towards MDCK cells in the concentration range 1-1000 μg/ml. Starting from a concentration of 2500 μg/ml, the composition according to the invention showed high toxicity towards the tested cells. The cytotoxic concentration values for 50% of cells (CC₅₀) of MDCK were 1652.4 μg/ml, 812.4 μg/ml, 1998.2 μg/ml, for elderberry extract, chokeberry extract and composition according to the invention, respectively.

### Evaluation of the Antiviral Activity of the Tested Extracts

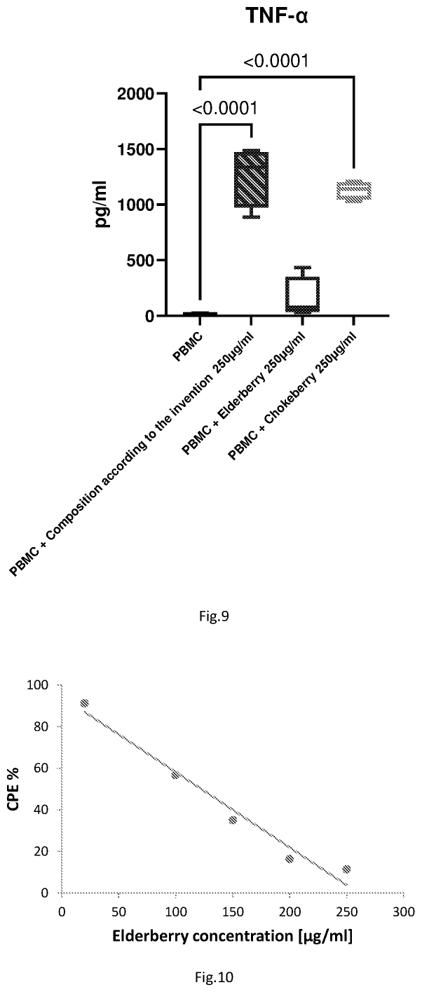

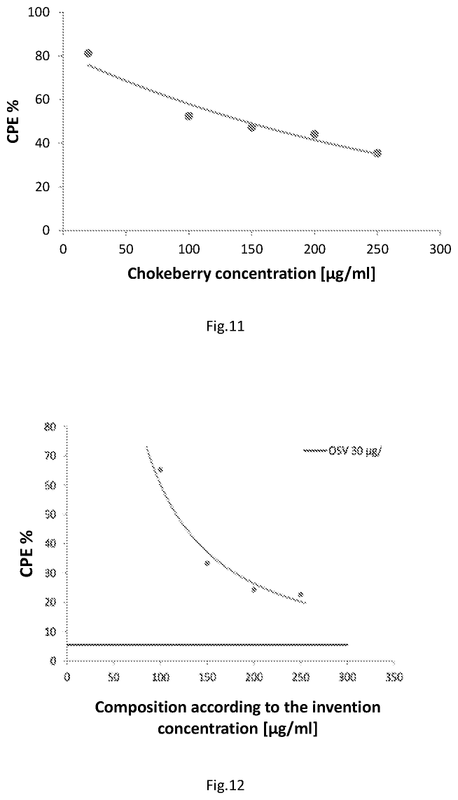

All extracts showed antiviral activity when administered after A/H1N1 virus infection. Elderberry extract at non-toxic concentrations inhibited A/H1N1 virus replication by more than 80%. Chokeberry in non-toxic concentrations inhibited A/H1N1 influenza virus replication by 60-70%, while the composition according to the invention in nontoxic concentrations inhibited A/H1N1 virus replication by 80%. The results are presented in FIG. 10-12.

### Determination of Selectivity Indices

The selectivity indices of the extracts against A/H1N1 virus (antiviral activity) are shown in Table 2.

TABLE 2
Antiviral activity of elderberry extracts, chokeberry extracts and
the composition according to the invention against A/H1N1 virus.

| | | | COMPOSITION |
|---|---|---|---|
| | ELDERBERRY | CHOKEBERRY | ACCORDING TO |
| | EXTRACT | EXTRACT | THE INVENTION |
| CC₅₀ [μg/ml] | 1652.4 | 812.4 | 1998.2 |
| IC₅₀ [μg/ml] | 122.5 | 102.3 | 116 |
| SI | 13.49 | 7.95 | 17.23 |

CC₅₀ (cytotoxic concentration)—the concentration of a preparation that is toxic to 50% of the cells in a culture compared to a control.
IC₅₀ (inhibitory concentration)—the concentration of the preparation that inhibits viral replication by 50% compared to the viral control.
SI (selectivity index)—selectivity index = CC50/IC50.

### Final Conclusions

1. The composition according to the invention showed very low cytotoxicity and the highest selectivity index compared to elderberry or chokeberry extracts.

2. The highest non-toxic dose of the composition according to the invention (250 μg/ml) inhibited the proliferation of the influenza virus in 80%. To obtain 80% inhibition of A/H1N1 by elderberry extract alone, concentrations above 200 μg/ml would have to be used. The content of elderberry and chokeberry extracts in the composition according to the invention is 2:1, which means that lower concentrations of elderberry with the addition of chokeberry allow maintaining the same high antiviral activity of the composition according to the invention as elderberry alone.

3. A proposed ratio (2:1) is ideal for the content of chokeberry extract, as increasing its content would not significantly affect the increase in antiviral activity of the composition according to the invention.

### EXAMPLE 4

### Anthocyanins, Polyphenols Content of the Composition According to the Invention

Composition according to the invention 10%
A. polyphenols=20,84%; anthocyanins=10,04%
B. polyphenols=21,75%; anthocyanins=10,42%
C. polyphenols=22,63%; anthocyanins=10,49%

Composition according to the invention 15%
A. Polyphenols=39,14%; anthocyanins=16,42%
B. Polyphenols=30,56%; anthocyanins=15,29%

Composition according to the invention 25%
A. polyphenols=49,77%; anthocyanins=28,48%

### EXAMPLE 5

Ativirial Activity of Fruit Extracts from Elderberry, Chokeberry and Composition According to the Invention Against Betacoronavirus Hcov-Oc43

### Research Methodology

### Tested Extracts

Standardized extract of elderberry fruits (Sambucus nigra), standardized extract of chokeberry fruits (Aronia melanocarpa) and a composition according to the invention comprising standardized extract of chokeberry and standardized extract of elderberry fruits. A starting concentration of the extracts, 10 mg/ml was prepared in 50% DMSO freshly before each experiment. Subsequently, the extracts were diluted in culture medium to appropriate concentrations. The final DMSO concentration in cells was ≤5% (non-toxic concentration for cells).

Cell lines HCT-8 colon cancer cell line [HRT-8] (ATCC. CCL-244™). Cells were cultured in RPMI-1640 medium supplemented with 10% horse serum.

### Tested Virus

Human HCoV-OC43 virus (ATCC. VR-1558™—betacoronavirus 1).

### Evaluation of the Cytotoxicity of Tested Preparations

The cytotoxicity of the tested preparations was evaluated by treating 24 h cultures of HCT-8 cells with selected doses, dissolved in RPMI-1640 culture medium without FBS (chokeberry extract: 7,8-250 μg/ml, elderberry extract: 62,5-1000 μg/ml, composition according to the invention: 15,63-1000 μg/ml). The cells were then incubated at 37° C., 5% CO₂. After 96 h of incubation, control microscopic readings of changes in cell morphology, indicating cytotoxicity (CTE, cytotoxic effect), were performed on a scale: 0— no cytotoxicity; 1— up to 25%; 2-50%; 3-75%; 4-100% cytotoxicity. To study the effect of the preparations on viral replication, 3 doses were selected for each preparation at which cell viability does not fall below 75%.

### Determination of the Effect of Preparations on Betacoronavirus 1 Replication

The effect of the preparations on viral replication was evaluated at a dose of 100 TCID50/ml after virus entry into cells (pre-incubation of cells with virus and subsequent culture with the tested preparation). A 24-hour culture of HCT-8 cells was incubated with virus in RPMI-1640 medium, in a 33° C., 5% CO₂. for 90 minutes. The culture was then thoroughly rinsed of virus, followed by application of selected doses of preparations from the range:
Chokeberry extract: 7,8-31,25 μg/ml
Elderberry extract: 62,5-250 μg/ml
Composition according to the invention: 15,63-125 μg/ml

prepared in RPMI-1640 medium without FBS and incubated for 96 hours at 33° ° C., 5% CO₂. After this time, virus titres were read under a microscope.

In addition, an immunoperoxidase test was also performed to confirm microscopic readings of CPE caused by HCoV-OC43 virus. This test allows the detection of even very low titres of HCoV-OC43 virus when the typical cytopathic effect (CPE) is not visible. After 96 h incubation of cultures treated with preparations and HCoV-OC43 virus, staining was performed with primary antibodies against HCoV-OC43 virus (mouse anti-coronavirus monoclonal antibodies) and secondary antibodies—goat anti mouse IgG HRP. The colour reaction was then performed with DAB/H₂O₂ and brown stained cells containing HCoV-OC43 virus were observed under the microscope.

The Staining Effect was Assigned to a Scale of 0-4, where:
0— no colouring
1— less than 25% of cells stained
2—25% to 50% of cells stained
3— more than 50 to 75% cells stained
4— more than 75 to 100% cells stained

### Results

Determination of Non-Toxic Concentrations of Extracts from Chokeberry, Elderberry and Composition According to the Invention

In the case of both chokeberry extract, elderberry extract and the composition according to the invention, the % of live HCT-8 cells decreased with increasing concentration of the preparations. To study the effect of the preparations on HCoV-OC43 virus replication, concentrations at which the number of viable cells in culture was at least 80% were applied.

### Effect of the Tested Preparations on HCoV-OC43 Virus Replication

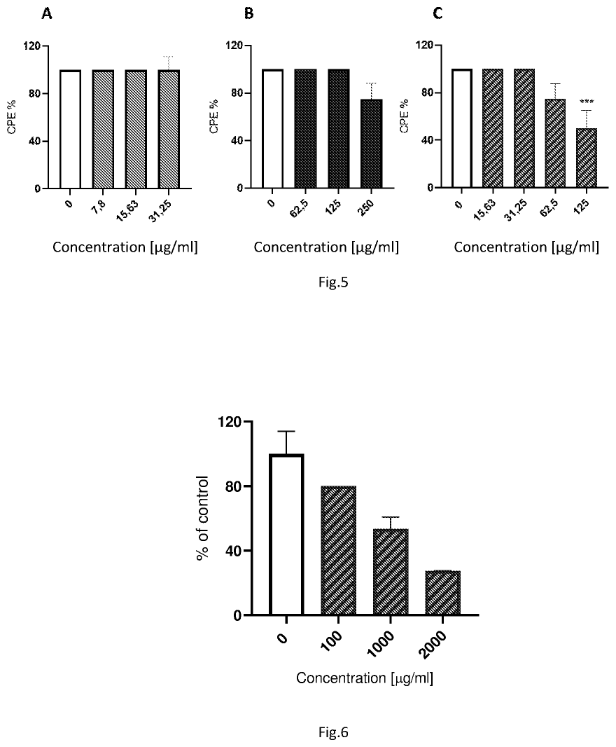

Both chokeberry extract (FIG. 5A) and elderberry extract (FIG. 5B) in the tested concentrations range did not show antiviral activity against HCoV-OC43 virus. On the other hand, the composition according to the invention showed antiviral activity against HCoV-OC43 virus, inhibiting its replication after entering cells by about 50% (at a concentration of 125 μg/ml) (FIG. 5C).

### EXAMPLE 6

### Evaluation of the Inhibitory Effect of Composition According TO Invention on ACE2 and SARS-COV2 RBD Binding In Vitro

### Research Methodology

Evaluation of inhibition of SARS-COV2 binding of RBD (receptor-binding domain) and hACE2 (human angiotensin-converting enzyme 2) was performed using the COVID-19 Spike—ACE2 kit (CoV-SACE2-1, RayBiotech Inc, https://www.raybiotech.com/covid-19-spike-ace2-binding-test-kit) according to the protocol provided by the manufacturer. The composition according to the invention was prepared in three concentrations (100, 1000, 2000 μg/ml) and the inhibitory potential for each concentration was evaluated in duplicate. The extracts analyzed were mixed with recombinant hACE2 protein, added to an ELISA plate coated with recombinant RBD of SARS-COV2 protein and incubated overnight at 4° C. Unbound ACE2 was removed by washing, and binding was assessed by reaction of an anti-ACE2 antibody conjugated to HRP (horseradish peroxidase) with 3,3′,5,5′-tetramethylbenzidine (TMB). Absorbance at 450 nm was measured with a PerkinElmer reader.

### Results

Analysis of the ACE2-SARS COV2 RBD binding assay in vitro showed a concentration dependent inhibitory effect of the composition according to the invention (FIG. 6).

### LITERATURE

1.Aksoy, B. A., Aksoy, P., Wyatt, M., Paulos, C. M., & Hammerbacher, J. (2018). PBMC isolation from buffy coat or whole blood PBMC cryopreservation. Protocols.io https://dx.doi.org/10.17504/protocols.io.qu2dwye.
2.Anton, A. M., Pintea, A. M., Rugină, D. O., Sconta, Z. M., Hanganu, D., Vlase, L., & Benedec, D. (2013). Preliminary studies on the chemical characterization and antioxidant capacity of polyphenols from Sambucus SP. Digest Journal of Nanomaterials and Biostructures, 8(3): 973-980.
3. Appel, K., Meiser, P., Milln, E., Collado, J. A., Rose, T., Gras, C. C., Carle, R., Muoz, E (201S). Chokeberry (Aronia melanocarpa (Michx.) Elliot) concentrate inhibits NF-_{K}B and synergizes with selenium to inhibit the release of pro-inflammatory mediators in macrophages. Phytotherapy, 105-73-82.
4. Barak, V., Halperin, T., & Kalickman, I. (2001). The effect of Sambucol, a black elderberry-based natural product, on the production of human cytokines: I. Inflammatory cytokines. European Cytokine Network, 12(2): 290-296.
5. Bonarska-Kujawa, D., Pruchnik, H., Oszmiański, J., Sarapuk, J., & Kleszczyńska, H. (2011). Changes Caused by Fruit Extracts in the Lipid Phase of Biological and Model Membranes. Food Biophysics, 6(1): 58-67.
6. Chen, C., Zuckerman, D. M., Brantley, S., Sharpe, M., Childress, K., Hoiczyk, E., & Pendleton, A. R. (2014). Sambucus nigra extracts inhibit infectious bronchitis virus at an early point during replication. BMC Veterinary Research, 10(24).
7. Glatthaar-Saalmüller, B., Fal, A. M., Sch.nknecht, K., Conrad, F., Sievers, H., & Saalmüller, A. (2015). Antiviral activity of an aqueous extract derived from Aloe arborescens Mill. against a broad panel of viruses causing infections of the upper respiratory tract. Phytomedicine, 22(10): 911-920.
8. Hawkins, J., Baker, C., Cherry, L., Dunne, E. (2019). Black elderberry (Sambucus nigra) supplementation effectively treats upper respiratory symptoms: A metaanalysis of randomized, controlled clinical trials. Complement Ther Med., 42: 361-365.
9. Ho, G. T. T., Brunlich, M., Austarheim, I., Wangensteen, H., Malterud, K. E., Slimestad, R., & Barsett, H. (2014). Immunomodulating activity of Aronia melanocarpa polyphenols. Int J Mol Sci., 15(7): 11626-11636.
10. Kinoshita, E., Hayashi, K., Katayama, H., Hayashi, T., Obata, A. (2012). Anti-influenza virus effects of elderberry juice and its fractions. Biosci Biotechnol Biochem, 76(9): 1633-8.
11. Kong, F. (2009). Pilot clinical study on a proprietary elderberry extract: efcacy in addressing infuenza symptoms. J Pharmacol Pharmacokinet., 5: 32-43.
12. Krawitz, C., Mraheil, M. A., Stein, M., Imirzalioglu, C., Domann, E., Pleschka, S., & Hain, T. (2011). Inhibitory activity of a standardized elderberry liquid extract against clinically-relevant human respiratory bacterial pathogens and influenza A and B viruses. BMC Complement Altern Med., 11(1), 16. doi.10.1186/1472-6882-11-16.
13. Lin. P. Hwang, E., Ngo, H. T. T., Seo, S. A., Yi, T. H. (2019). Sambucus nigra L. ameliorates UVB-induced photoaging and inflammatory response in human skin keratinocytes. Cytotechnology, 71(5): 1003-1017.
14. Macdonald, S. J., Watson, K. G., Cameron, R., Chalmers, D. K., Demaine, D. A., Fenton, R. J., Gower, D., Hamblin, J. N., Hamilton, S., Hart, G. J., Inglis, G. G., Jin, B., Jones, H. T., McConnell, D. B., Mason, A. M., Nguyen, V., Owens, I. J., Parry, N., Reece, P. A., Shanahan, S. E., Smith, D., Wu, W. Y., Tucker, S. P., (2004). Potent and long-acting dimeric inhibitors of influenza virus neuraminidase are effective at a once weekly dosing regimen. Antimicrob. Agents Chemother., 48: 4542-4549.
15. Martin, D. A., Taheri, R., Brand, M. H., Draghi, A., Sylvester, F. A., Bolling, B. W. (2014). Anti-inflammatory activity of aronia berry extracts in murine splenocytes. J. Funct. Foods, 8: 68-75.
16. Morag, A., Mumcuoglu, M., Baybikov, T., Schelsinger, M., Zakay-Rones, Z. (1997) Inhibition of sensitive and acyclovir-resistant HSV-1 strains by an elderberry extract in vitro. J Z. Phytother, 25: 97-98.
17. Ohgami, K., Ilieva, I., Shiratori, K., Koyama, Y., Jin, X H., Yoshida, K., Kase, S., Kitaichi, N., Suzuki, Y., Tanaka, T., Ohno, S. (2005). Anti-inflammatory effects of aronia extract on rat endotoxin-induced uveitis. Invest Ophthalmol Vis Sci., 46(1): 275-281.
18. Park, S., Kim, J. I., Lee, I., Lee, S., Hwang, M. W., Bae, J. Y., Heo, J., Kim, D., Han, S. Z., & Park, M. S. (2013). Aronia melanocarpa and its components demonstrate antiviral activity against influenza viruses. Biochem Biophys Res Commun., 440(1): 14-19.
19. Roschek, B., Fink, R. C., McMichael, M. D., Li,D., & Alberte, R. S. (2009). Elderberry flavonoids bind to and prevent H1N1 infection in vitro. Phytochemistry, 70(10): 1255-1261.
20. Sekizawa, H., Ikuta, K., Mizuta, K., Takechi, S., & Suzutani, T. (2013). Relationship between polyphenol content and anti-influenza viral effects of berries. J Sci Food Agric., 93(9): 2239-2241.
21. Swaminathan, K., Dyason, J. C., Maggioni, A., von Itzstein, M., Downard, K. M. (2013). Binding of a natural anthocyanin inhibitor to influenza neuraminidase by mass spectrometry. Anal Bioanal Chem., 405(20): 6563-6572.
22. Tiralongo, E., Wee, S. S., & Lea, R. A. (2016). Elderberry supplementation reduces cold duration and symptoms in air-travellers: A randomized, double-blind placebocontrolled clinical trial. Nutrients, 8(4), 182. doi.10.3390/nu8040182.
23. Torabian, G., Valtchev, P., Adil, Q., & Dehghani, F. (2019). Anti-influenza activity of elderberry (Sambucus nigra). Journal of Functional Foods, 54: 353-360.
24. Ulbricht, C., Basch, E., Cheung, L., Goldberg, H., Hammerness, P., Isaac, R., Khalsa, K. P. S., Romm, A., Rychlik, I., Varghese, M., Weissner, W., Windsor, R. C., & Wortley, J. (2014). An evidence-based systematic review of elderberry and elderflower (Sambucus nigra) by the natural standard research collaboration. J Diet Suppl., 11(1): 80-120.
25. Uncini M. R. E., Zaccaro, L., & Tomei, P. E. (2005). Antiviral activity in vitro of Urtica dioica L., Parietaria diffusa M. et K. and Sambucus nigra L. J Ethnopharmacol., 98(3): 323-327.
26. Zakay-Rones Z., Varsano, N., Zlotnik, M., Manor, O., Regev, L., Schlesinger, M., & Mumcuoglu, M. (1995). Inhibition of Several Strains of Influenza Virus in Vitro and Reduction of Symptoms by an Elderberry Extract (Sambucus nigra L.) during an Outbreak of Influenza B Panama. J Altern Complement Med, (4): 361-369.
27. Zakay-Rones, Z., Thom, E., Wollan, T., Wadstein, J. (2004). Randomized study of the efficacy and safety of oral elderberry extract in the treatment of influenza A and B virus infections. J Int Med Res., 32(2): 132-40.
28. Zielińska-Wasielica, J., Olejnik, A., Kowalska, K., Olkowicz, M., Dembczynski, R. (2019). Elderberry (Sambucus nigra L.) Fruit Extract Alleviates Oxidative Stress, Insulin Resistance, and Inflammation in Hypertrophied 3T3-LI Adipocytes and Activated RAW 264.7 Macrophages. Foods, 8(8): 326. doi: 10 3390/foods8080326.

1. A pharmaceutical composition comprising:
a mixture of elderberry fruit extract (Sambucus nigra) and chokeberry fruit extract (Aronia melanocarpa),
wherein a mixture of elderberry fruit extract (Sambucus nigra) and chokeberry extract (Aronia melanocarpa) contains anthocyanins 10-30% and polyphenols 15-50%, and
wherein a ratio of extracts of elderberry (Sambucus nigra) and chokeberry (Aronia melanocarpa) in the composition is 2:1 to 1:2,
wherein the pharmaceutical composition is for stimulating immunity, enhancing immunity or both.

2. The pharmaceutical composition of claim 1, further comprising a pharmaceutical excipient or diluent or carrier.

3. The pharmaceutical composition of claim 1, wherein the pharmaceutical composition is for oral administration and is in a form selected from a group consisting of; tablets, capsules, and a solution.

4. The pharmaceutical composition of claim 1 for use in the manufacture of dietary supplements for use in supportive therapy for the treatment of viral diseases.

5. The pharmaceutical composition of claim 4, wherein it the pharmaceutical composition is applicable for use in influenza and/or influenza-like infections.

6. The pharmaceutical composition of claim 4, wherein the pharmaceutical composition it has an immunostimulatory application.

7. A preparation comprising the pharmaceutical composition of claim 1 for use for antiviral treatment.

8. The preparation of claim 7, wherein the preparation is applied orally.

9. The preparation of claim 7, wherein preparation it is used as a food supplement.

10. The pharmaceutical composition of claim 5, for use against a human betacoronavirus.

11. A method of using the pharmaceutical composition of claim 1 for one of the following:
a. use in the manufacture of dietary supplements for use in supportive therapy for the treatment of viral diseases;
b. use in influenza and/or influenza-like infections;
C. for immunostimulation; and
d. use against a human betacoronavirus.

12. The preparation of claim 7, for use against a human betacoronavirus.

13. A method of using the preparation of claim 7, for use as a food supplement.
